(19) United States
(12) Patent Application Publication
(10) Pub. No.: US 2021/0169541 A1
(43) Pub. Date: Jun. 10, 2021

(54) SEAL FOR APPLICATION OF BONE CEMENT

(71) Applicant: Heraeus Medical GmbH, Wehrheim (DE)

(72) Inventors: Edgar WÜST, Wehrheim (DE); Anna SPEHL, Wehrheim (DE)

(73) Assignee: Heraeus Medical GmbH, Wehrheim (DE)

(21) Appl. No.: 17/110,960

(22) Filed: Dec. 3, 2020

(30) Foreign Application Priority Data
Dec. 5, 2019 (EP) 19213729.7

(51) Int. Cl. A61B 17/88

(52) U.S. Cl. CPC A61B 17/8808; A61B 17/8816

(57) ABSTRACT

One aspect relates to a seal for sealing a bone canal opening during the application of bone cement into the cancellous bone of a bone canal. The aspect also relates to a device for applying bone cement into the cancellous bone of a bone canal including the seal according to one embodiment, and a method for applying bone cement by using the device according to the embodiment.

### CROSS-REFERENCE TO RELATED APPLICATION

This Utility patent application claims priority to European Application No. 19213729.7 filed on Dec. 5, 2019, which is incorporated herein by reference.

### TECHNICAL FIELD

One embodiment relates to a seal for sealing a bone canal opening during the application of bone cement into the cancellous bone of a bone canal. One embodiment also relates to a device for applying bone cement into the cancellous bone of a bone canal including the seal according to one embodiment, and a method for applying bone cement by using the device according to one embodiment.

### BACKGROUND

Total arthroplasty is a widely applied procedure in orthopaedics. It involves the removal of infected tissue, in particular bone tissue, and its replacement by artificial prostheses made of metal or plastics. Usually, the prostheses are affixed to the remaining healthy bone tissue by using bone cement. In particular, during the fixation of hip prostheses in the appropriately prepared medullary bone canal of the femur, the high mechanical load is associated with a risk of the prosthesis loosening, usually at the transition of bone cement to bone. In order to attain improved adhesion between bone cement and bone, the bone cement is applied into the bone canal at elevated application pressure. This leads to at least partial penetration of the bone cement into the cancellous bone, which would not be the case due to the high viscosity of the bone cement if the application pressure were too low, and thus leads to improved bonding of bone cement to bone.

The elevated application pressure is attained through the use of an application device for bone cement that is fitted with a seal. The bone cement is applied from the application device into the bone canal, whereby the seal appropriately seals the bone canal with respect to the ambient atmosphere during the application process such that the application proceeds at a sufficient application pressure to effect penetration of the bone cement into the cancellous bone.

Efforts are being undertaken to provide devices by means of which bone cement can be applied into a bone canal at a sufficient application pressure in order to effect penetration of the bone cement into the cancellous bone of the bone. The devices, in particular seals for the devices, are described, for example, in U.S. Pat. No. 4,815,454 A and 6,017,350 A.

U.S. Pat. No. 4,338,925 A addresses mainly an application device and a pressure application method for bone cement, wherein a multitude of different attachments for the application device is used. Inter alia, a seal, consisting of an adapter and a pressure holder, is described. The adapter consists of polyethylene, is screwed onto the end of the application device, and comprises a tube-shaped extension. The pressure holder consists of silicon and is conical in shape (cf. FIG. 4).

One of the disadvantages of the seal is that for sealing of the bone canal for the application of pressure the seal needs to penetrate deeply into the bone canal. In particular in the case of revision surgeries, in which the bone canal is widened even more due to the removal of substantial amounts of bone tissue, the penetration depth of the seal is significantly larger yet. The penetration depth of the seal being too large has multiple disadvantages. Firstly, the bone canal cannot be filled completely with bone cement as the area facing the application device remains unfilled, at least in part, and needs to be filled in a further procedural process not involving the use of the seal. Moreover specifically the area of the bone canal facing the application device is crucial for successful pressure application of the bone cement. In this area, the bone includes the so-called cancellous bone, a sponge-like bone structure that is the actual target of pressure application. The purpose of the application of pressure is to fill the cancellous bone and attain the resulting improvement of the adhesion between bone cement and bone, which is not available at insufficient application pressure due to the high viscosity of the bone cement. If areas of the cancellous bone are unavailable to the bone cement during the pressure application procedure, the adhesion of bone cement to bone is reduced and thus the mechanical stability of the prosthesis to be affixed with the bone cement is reduced as well. Another disadvantage is the improved risk of the seal breaking off the application device if there is any canting within the bone canal. The application of pressure requires the user to apply a high contact pressure with a resulting large mechanical stress on the seal against the bone canal, which is not always easily accessible during the operation for steric reasons, which increases the risk of the seal breaking off. Moreover, due to the presence of various body fluids, such as, for example, blood, there is a risk of the seal inadvertently slipping off the bone canal, which increases the risk of it breaking off even further.

For these and other reasons there is a need for the present embodiment.

### SUMMARY

It is an object of one embodiment to overcome, at least in part, one or more of the disadvantages resulting according to the prior art.

Specifically, one embodiment is based on the goal to provide a seal which permits at least partial filling of the cancellous bone of a bone with bone cement. The seal is to enable essential complete filling of a bone canal in a single filling process. The seal is to have high mechanical strength. The seal is to have a low risk of it breaking off during canting.

It is an object of one embodiment to provide a device for the application of bone cement into the cancellous bone of a bone canal by using which at least a part of the objects described above is solved at least in part.

It is an object of one embodiment to provide a method by using which at least a part of the object described above is solved at least in part.
[1] A seal for sealing a bone canal opening during the application of bone cement into the cancellous bone of a bone canal,
   characterised in that
   the seal comprises a concave sealing surface for sealing the bone canal.
[2] The seal according to embodiment 1, characterised in that the seal comprises a seal canal, whereby the seal canal can be reversibly connected in form-fitting and/or force-locking manner to a dispensing opening of a device providing the bone cement in order to apply the bone cement from the device out of a seal opening at an end of the seal canal that faces the sealing surface into the bone canal.
[3] The seal according to embodiment 1 or 2, characterised in that the seal opening is arranged in the centre of the sealing surface.
[4] The seal according to embodiment 2 or 3, characterised in that the sealing surface comprises a design height, whereby a maximum design height is evident adjacent to a seal opening area and the design height decreases from the seal opening area in the direction of the edges of the sealing surface according to a concave function.
[5] The seal according to embodiment 4, characterised in that the concave function comprises a largest slope adjacent to the seal opening area, and in that the slope decreases steadily in the direction of the edges of the sealing surface.
[6] The seal according to embodiment 4 or 5, characterised in that the maximum design height of the sealing surface is in the range of 5 to 20 mm, preferably of 8 to 17 mm, more preferably of 10 to 15 mm.
[7] The seal according to any of the preceding embodiments, characterised in that the sealing surface comprises an oval cross-sectional surface having a length in the range of 60 to 80 mm and a width in the range of 25 to 45 mm.
[8] The seal according to any one of the preceding embodiments, characterised in that the seal comprises a plastic material, in particular a thermoplastic resin, having a Shore hardness of 25 to 50 ShoreA, preferably consists of a plastic material, in particular a thermoplastic resin, having a Shore hardness of 25 to 50 ShoreA.
[9] A device for the application of bone cement into the cancellous bone of a bone canal, comprising
   a container, in which a bone cement can be stored;
   whereby the container comprises, on one end, a dispensing side with a dispensing opening protruding from the dispensing side for disposing the bone cement from the container into the bone canal;
   characterised in that
   the dispensing opening is reversibly connected in form-fitting and/or force-locking manner to a seal according to any one of the embodiments 1 to 8 for sealing a bone canal opening in order to apply the bone cement into the bone canal at sufficient application pressure.
[10] The device according to embodiment 9, characterised in that the seal is arranged on an external surface of a dispensing side, at least partly, in form-fitting manner.
[11] A device for the application of bone cement into the cancellous bone of a bone canal, comprising
   a container, in which a bone cement can be stored;
   whereby the container comprises, on one end, a dispensing side with a dispensing opening protruding from the dispensing side for disposing the bone cement from the container into the bone canal;
   a seal for sealing a bone canal opening in order to apply the bone cement into the bone canal at a sufficient application pressure;
   whereby the seal is reversibly connected in form-fitting and/or force-locking manner to the dispensing opening;
   characterised in that
   the seal is arranged on an external surface of a dispensing side, at least partly, in form-fitting manner.
[12] The device according to embodiment 9 to 11, characterised in that the application pressure is sufficient to allow the bone cement to penetrate into the cancellous bone by at least 2 mm, in particular between 3 to 5 mm deep.
[13] The device according to any one of the embodiments 9 to 12, characterised in that the seal comprises a rear side surface opposite from the sealing surface, whereby the rear side surface is arranged in form-fitting manner on the external surface of the dispensing side.
[14] The device according to embodiment 13, characterised in that at least 20 area-% of the rear side surface are arranged on the external surface of the dispensing side.
[15] The seal according to embodiment 13 or 14, characterised in that the rear side surface is arranged in a circular ring shape on the external surface of the dispensing side.
[16] A method for applying bone cement into the cancellous bone of a bone canal by means of a device according to any one of the embodiments 9 to 15, comprising at least the following steps of:
   a) providing the bone cement inside the device;
   b) pressing the device onto a bone canal opening such that the bone canal is sealed by a seal during the application of the bone cement;
   c) dispensing the bone cement from the device into the bone canal;
   whereby the seal is being pressed essentially onto a surface of the bone and/or onto a cut surface of the bone.
[17] The method according to embodiment 16, characterised in that the bone canal is being filled essentially completely with bone cement in step c).

### BRIEF DESCRIPTION OF THE DRAWINGS

Embodiments are illustrated further by examples in exemplary manner in the following. The invention shall not be limited to the examples.

In the figures,

FIG. 1 illustrates a schematic cross-section of a seal for sealing a bone canal;

FIG. 2 illustrates a top view onto a sealing surface of the seal from FIG. 1;

FIG. 3 illustrates a top view onto a rear side surface of the seal from FIG. 1;

FIG. 4 illustrates a schematic cross-section of a device for applying bone cement including the seal from FIG. 1; and

FIG. 5 illustrates a method for applying bone cement into the cancellous bone of the bone canal.

### DETAILED DESCRIPTION

In the following Detailed Description, reference is made to the accompanying drawings, which form a part hereof, and in which is illustrated by way of illustration specific embodiments in which one embodiments may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present embodiments. The following detailed description, therefore, is not to be taken in a limiting sense, and the scope of the present embodiments are defined by the appended claims.

It is to be understood that the features of the various exemplary embodiments described herein may be combined with each other, unless specifically noted otherwise.

### General Information

Ranges specified in the present description include the values specified as the limits. A specification of “in the range of X to Y” with regards to a parameter A therefore means that A can assume values X, Y, and values between X and Y. Accordingly, ranges of a parameter A limited on one side in the way of “up to Y” include values equal to Y and less than Y.

Some of the features described herein are linked to the term, “essentially”. The term “essentially” shall be understood to mean that a mathematically exact interpretation of terms such as “superimposition”, “perpendicular”, “diameter” or “parallelism” can never be evident exactly, but only within certain error tolerances of production technology for real conditions and production technologies. For example, “essentially parallel axes” include an angle of 85 degrees to 95 degrees with respect to each other and “essentially equal volumes” comprise a deviation of up to 5 volume-%. A “device consisting essentially of plastic material” comprises, for example, a plastic fraction of <95 to >100 weight-%. An “essentially complete filling of a volume B” comprises, for example, a filling of <95 to >100 volume-% of the total volume of B.

One embodiment relates to a seal for sealing a bone canal opening during the application of bone cement into the cancellous bone of a bone canal, characterised in that the seal includes a concave sealing surface for sealing the bone canal.

A seal is a fluid-conducting element that can be reversibly attached to a device in order to apply a bone cement from the device into a bone canal at appropriately high application pressure such that the bone cement is being introduced, at least in part, into the cancellous bone of the bone canal. In this context, the seal connects the device and the bone canal in order to allow the bone cement to be applied from the device into the bone canal, while simultaneously preventing any leakage of the bone cement from the bone canal opening. Progressive application of the bone cement into the bone canal thus leads to a sufficiently high application pressure that effects at least partial introduction of the bone cement into the cancellous bone of the bone canal. Due to the dough-like, highly viscous consistency of the bone cement, a minimal level of application pressure is required that depends on the actual nature of the bone cement. If the minimal level is not attained, the bone cement is not introduced into the cancellous bone. The minimal level of the application pressure is equivalent to a sufficient application pressure.

The bone cement used for arthroplasty has a dynamic viscosity, for example 2 to 6 minutes after being mixed at room temperature (approx. 20 to 22° C.) in the range of 100 to 500 Pa*s.

A sufficient application pressure shall be understood to mean that progressive conveyance of the bone cement into the bone canal with the bone canal opening being sealed by the seal enables the introduction of bone cement into the cancellous bone of the bone by at least 2 mm, in one embodiment between 3 and 5 mm.

At least partial introduction of the bone cement into the cancellous bone of a bone canal, in one embodiment of the femoral bone canal, affords an increased adhesion force between the corresponding bone and the bone cement and thus improved fixation of a prosthesis, in one embodiment a hip prosthesis, affixed with the bone cement.

For application, the device fitted with the seal is pressed appropriately against the bone canal such that a sealing surface of the seal facing the bone canal seals the bone canal and enables an application of the bone cement from the device into the bone canal at sufficient application pressure.

The sealing surface of the seal is designed to be concave in shape. A concave sealing surface permits, firstly, reliable sealing of the bone canal and, secondly, essentially complete filling of the bone canal with bone cement at sufficient application pressure, in one embodiment complete filling of the bone canal with bone cement in a single application process. Due to its concave design, the sealing surface is essentially not in contact with the cancellous bone during the application of the bone cement, but rather is situated at a distance therefrom, which allows the bone cement to have free access to the cancellous bone and thus be introduced into the cancellous bone.

The concave sealing surface can take different designs.

In one embodiment, the sealing surface is shaped as a spherical shell, for example as a semi-spherical cell, whereby the spherical shell can include a circular, elliptical or irregular-shaped cross-section. In this context, the circular ring-shaped external surface of the spherical shell is being pressed against the bone such that the bone canal is sealed with respect to the ambient atmosphere while the bone cement is being applied through the corresponding opening of the spherical shell into the bone canal. Any excess of bone cement remaining in the spherical shell-shaped seal after the application can be removed by the surgeon, for example by hand, after the bone canal is being filled.

Another embodiment of the seal is characterised in that the seal includes a seal canal, whereby the seal canal can be reversibly connected in form-fitting and/or force-locking manner to a dispensing opening of a device providing the bone cement in order to apply the bone cement from the device out of a seal opening at an end of the seal canal that faces the sealing surface into the bone canal.

The seal includes a seal canal. A seal canal shall be understood to be a fluid-conducting, tube-like connection in the axial extension of the seal that allows for conveying the bone cement from a side facing the device in the direction of the sealing surface of the seal facing the bone canal. On the side of the sealing surface, the seal canal merges into the seal opening.

For conveying the bone cement, the seal canal is connected in form-fitting and/or force-locking manner to the dispensing opening of the device. A first embodiment of the seal canal includes an internal thread that acts in concert with an external thread of the dispensing opening in order to connect the bone canal, and thus the seal as well, to the dispensing opening. A further embodiment of the seal canal includes, at least over regions thereof, an internal cross-section that corresponds essentially to an external diameter of the dispensing opening in order to thus connect the seal canal to the dispensing opening by using a form-fit and/or force-lock. A further embodiment of the seal includes a connecting element, for example a thread or a bayonet closure, connecting the seal to the device such that the seal canal touches essentially in form-fitting manner against the dispensing opening and enables the conveyance of the bone cement through the seal canal. In a further embodiment, the cross-section of the seal canal is constant along the entire axial extension of the seal canal. In a further embodiment, the seal canal is conical in shape, whereby, in one embodiment the cross-section of the seal canal increases in the direction of the seal opening.

The seal canal can follow any course through the seal. In one embodiment, the seal canal follows an essentially straight axial course through the seal.

The seal opening can be placed at different positions of the sealing surface.

A further embodiment of the seal is characterised in that the seal opening is arranged in the centre of the sealing surface.

A seal opening being placed in the centre shall be understood to mean that the seal opening is situated at the centre of an area equivalent to a projection of the sealing surface, rather than in the edge area of the sealing surface. In case of the sealing surface being rotationally symmetrical in design, this would correspond to the area of the rotation axis.

Arranging the seal opening in the centre facilitates the use of the seal by the surgeon, since rotation of the device about a longitudinal axis during the application, or at least during the sealing of the bone canal, results in no or only little spatial shift of the seal opening with respect to the bone canal.

Another embodiment of the seal is characterised in that the sealing surface includes a design height, whereby a maximum design height is evident adjacent to a seal opening area and the design height decreases from the seal opening area in the direction of the edges of the sealing surface according to a concave function.

A design height shall be understood to be an axial extension of the sealing surface along a longitudinal axis of the seal, whereby the edges, or at least the edge closest to the device in axial extension, define a plane that is situated perpendicular to the longitudinal axis of the seal, against which the design height is being determined.

The seal includes a seal opening area. A seal opening area directly borders the seal opening radially and surrounds the seal opening, at least over regions thereof, in one embodiment completely, in radial extension. The seal opening area extends essentially perpendicular to a longitudinal axis of the seal and transitions into the sealing surface. The seal opening area has a radial extension about the seal opening in the range of 1 mm to 2 cm.

One advantage of this embodiment is that, as a result, the area of the seal opening is introduced into the bone canal, although only to a small degree due to the concave shape of the sealing surface, during the sealing and application of the bone cement. On the one hand, this makes it easier for the surgeon to press the seal onto the bone canal in a perfect fit by using a haptic feedback during the introduction of the sealing opening into the bone canal. On the other hand, this reduces the danger of the seal inadvertently slipping out of the opening of the bone canal during the application, since the design height of the sealing surface makes slipping more difficult.

The decrease in the design height of the sealing surface according to a concave function ensures free access of the bone cement into the cancellous bone of the bone canal in the area of the opening of the bone canal and, simultaneously, a small penetration depth of the sealing opening into the bone canal. Concurrently, the shape of the seal allows the surgeon to securely and stably press the device onto the bone canal, even at high contact pressure. Moreover, the bone canal can be filled essentially completely with bone cement in one procedural process, without placing down the device and removal of the seal.

In order for the free access of the bone cement to the cancellous bone of the bone to be facilitated even more and, simultaneously, the penetration depth of the seal opening being as small as possible, a further embodiment of the seal is characterised in that the concave function includes the largest slope adjacent to the seal opening area, and in that the slope decreases steadily in the direction of the edges of the sealing surface.

In one embodiment, the concave function includes the largest slope directly adjacent to the seal opening area. In further embodiments, the concave function includes the largest slope not directly adjacent to the seal opening area, but the largest slope is at a distance from the seal opening area, in one embodiment from an edge of the seal opening area, of up to 5 mm, in one embodiment up to 3 mm, in one embodiment up to 1 mm. Regardless of whether the maximum slope of the concave function is directly adjacent to the seal opening area or at a distance from the seal opening area, in one embodiment at a distance from an edge of the seal opening area, the slope of the concave surface decreases steadily from the point of maximum slope in the direction of the edges of the sealing surface.

A further embodiment of the seal is characterised in that the maximum design height of the sealing surface is in the range of 5 to 20 mm, in one embodiment of 8 to 17 mm, in one embodiment of 10 to 15 mm.

One advantage of the maximum design height being in the range of 5 to 20 mm, in one embodiment of 8 to 17 mm, in one embodiment of 10 to 15 mm is that a well-balanced compromise between low penetration depth of the seal into the bone canal and concurrent good adhesion when the device is being pressed onto the bone canal is attained.

The sealing surface can include any cross-sectional shape. For example, the cross-sectional surfaces can be designed to be round.

A further embodiment of the seal is characterised in that the sealing surface includes an oval cross-sectional surface having a length in the range of 60 to 80 mm and a width in the range of 25 to 45 mm.

Oval, in one embodiment ovoid oval, cross-sectional surfaces are preferred in one embodiment due to the good shape conformance with the bone canals, which typically are oval in cross-section, and due to a good adaptability to bone canal openings of differing in size. Moreover, a contact surface of the sealing surface against the bone can be easily adapted to the existing shape of the bone canal opening through a rotation about the longitudinal axis of the seal. The length being in the range of 60 to 80 mm and the width being 25 to 45 mm allows for a use of the seal for a multitude of different opening cross-sections of bone canals, in one embodiment for bone canals including an enlarged bone canal opening in the course of a revision surgery.

The seal can be manufactured from a variety of materials. For good sealing of the bone canal, in one embodiment referring to bone canal openings which, for steric reasons, are accessible only with an unfavourable, non-axial contact angle or possess an irregular bone surface in the area of the bone canal opening, it is preferred for the seal to consist of a deformable material.

Another embodiment of the seal is characterised in that the seal includes a plastic material, in one embodiment a thermoplastic resin, having a Shore hardness of 25 to 50 ShoreA, in one embodiment consists of a plastic material, in one embodiment a thermoplastic resin, having a Shore hardness of 25 to 50 ShoreA.

The shore hardness of the plastic material being more than 50 ShoreA, in one embodiment for a non-axial contact angle against the bone canal opening, results in insufficient sealing of the bone canal due to the absence of a form fit between seal and bone such that a sufficient bone cement application pressure cannot be attained.

If on the other hand, the Shore hardness is less than 25 Shore A, the plastic material is too soft such that the seal might enable a form-fit with the bone canal opening, but simultaneously seals the bone canal insufficiently since the flexibility of the seal is too high. If the plastic material is too soft, an elevated application pressure presses the bone cement not into the cancellous bone, but past the seal out of the bone canal.

Examples of the plastic materials include plastics from the plastics classes of the silicones, thermoplastic elastomers (TPE) and/or thermoplastic vulcanizates (TPV), such as, for example, Santopren.

A further embodiment relates to a device for the application of bone cement into the cancellous bone of a bone canal, including a container, in which a bone cement can be stored, whereby the container includes, on one end, a dispensing side with a dispensing opening protruding from the dispensing side for dispensing the bone cement from the container into the bone canal, characterised in that the dispensing opening is reversibly connected in form-fitting and/or force-locking manner to a seal according to any of the preceding embodiments for sealing of a bone canal opening in order to apply the bone cement into the bone canal at sufficient application pressure.

The device includes a container. A container shall be understood to be a tubular vessel that includes an internal space and a container wall surrounding the internal space. The container possesses a cross-section perpendicular to a longitudinal axis. According to one embodiment, the cross-section of the container can take any shape. For example, the cross-section can be designed to be oval, square, pentagonal, hexagonal, irregular or circular.

The container is preferred to include a cylindrical geometry with a rotationally symmetrical axis with a round cross-section. The geometry allows for good handling by the user and the absence of edges reduces the risk of wedging during the use of the device. According to one embodiment, the container can consist of a broad range of materials or combinations of materials. For example, the container can consist of plastic material. In one embodiment, the plastic material is a transparent plastic material as this allows the user to check by eye the proper function of the device during a use thereof.

The container includes, on an end, in one embodiment on an axial end, a dispensing side that rounds off the container. The dispensing side includes a dispensing opening through which the bone cement can be conveyed from the internal space of the container, whereby the dispensing opening extends axially in the direction of the side of the dispensing side facing away from the internal space of the container. The protruding dispensing opening permits the reversible attachment of aids, such as for example a snorkel or a seal, which are useful or required during the application of the bone cement.

The attachment of the seal can be implemented in a variety of ways. In one embodiment, the dispensing opening includes an internal thread that acts in concert with an external thread of the seal in order to establish a form-fitting and/or force-locking connection. In a further embodiment, the dispensing opening includes an external thread that acts in concert with an internal thread of the seal in order to establish a form-fitting and/or force-locking connection. In a further embodiment, the dispensing opening and the seal are connected to each other by using a bayonet connection. In a further embodiment, the seal includes a recess that acts in concert in form-fitting and/or force-locking manner with the dispensing opening protruding from the dispensing side in order to attach the seal. In a further, preferred, embodiment, the internal diameter of the seal canal corresponds essentially to the external diameter of the dispensing opening, such that the seal can be connected to the dispensing opening in form-fitting and/or force-locking manner by inserting the dispensing opening into the seal canal.

In an embodiment of the device, the connected seal is arranged exclusively on the dispensing opening and is not in direct physical contact with further parts of the device.

A further embodiment of the devices characterised in that the seal is arranged on an external surface of a dispensing side in at least partly form-fitting manner.

The external surface of the dispensing side is the side of the dispensing side that faces the seal and faces away from the internal space of the container.

A form-fitting arrangement shall be understood to mean that the seal and the external surface of the dispensing side are in direct physical contact, at least over regions thereof. In the embodiment, the seal is in direct physical contact both with the dispensing side opening, for attaching the seal to the device, as well as with the external surface of the dispensing side.

One advantage of the seal touching against the dispensing side surface in form-fitting manner is that the seal possesses a high mechanical strength when pressed against the bone canal. Touching against the dispensing side provides for a more extensive distribution of force onto the seal when the device is pressed against the bone canal than would be the case upon attachment of the seal just in spots, for example if there is any physical contact exclusively with the dispensing opening. In one embodiment, the form-fit allows the connection between seal and device to be mechanically stressed to a lesser degree. This does not only increase the mechanical strength of the seal, but also the mechanical strength of the device as a whole, in one embodiment of the attachment site of the seal, such as, for example, the dispensing opening. This is advantageous in one embodiment upon canting of the seal in the bone canal which may cause the device to fracture due to the high requisite contact pressure against the bone if the mechanical stability is low. The increased mechanical strength of the seal and of the device as a whole allows the surgeon to exert a higher contact pressure against the bone canal, which allows for complete sealing of the bone canal opening, in one embodiment if the contact angle of the device against the bone is not perpendicular.

A further embodiment of the device is characterised in that the application pressure is sufficient to allow the bone cement to penetrate into the cancellous bone by at least 2 mm, in one embodiment between 3 to 5 mm deep.

A penetration depth of less than 2 mm leads to the bone cement, and the prosthesis, in one embodiment the hip prosthesis, fixed with the bone cement coming loose due to the stress of it being in use, in one embodiment during walking motions of the patient. This leads to secondary surgeries that could be prevented or at least delayed in time at a penetration depth of more than 2 mm.

If the area of the affixed prosthesis were to become infected directly postoperatively or over the course of time, the prosthesis needs to be replaced. This is done, inter alia, by completely removing the bone cement used for fixation along with the region of the cancellous bone into which the bone cement has penetrated. In order not to have to remove excessive fractions of the cancellous bone and to be able to use part of the residual cancellous bone again for improved adhesion of bone to bone cement in the subsequent fixation of the new prosthesis, it is preferred not to allow the bone cement to penetrate into the cancellous bone by more than 5 mm.

A further embodiment of the device is characterised in that the seal includes a rear side surface opposite from the sealing surface, whereby the rear side surface is arranged in form-fitting manner on the external surface of the dispensing side.

The form-fitting contact between the rear side surface and the external surface of the dispensing side can be implemented in a variety of ways. In an embodiment, at least 10 area-% of the rear side surface are arranged in form-fitting manner on the external surface of the dispensing side. Area-% of the rear side surface shall be understood to be the fraction of the rear side surface which touches against the dispensing side surface in form-fitting manner, relative to the total area of the rear side surface. The higher this fraction, the higher is the mechanical strength of the seal and thus of the device as a whole as well.

A further embodiment of the device is characterised in that at least 20 area-% of the rear side surface are arranged on the external surface of the dispensing side.

Up to 100 area-% of the rear side surface can be arranged on the external surface of the dispensing side.

The rear side surface, in one embodiment the part of the rear side surface arranged in form-fitting manner on the external surface of the dispensing side, can be implemented in a variety of ways. In an embodiment, the rear side surface is implemented to be planar. In a further embodiment, the entire rear side surface is implemented as negative form of the external surface of the dispensing side such that the entire rear side surface or at least a part of the rear side surface is arranged in form-fitting manner on the external surface of the dispensing side.

A further embodiment of the device is characterised in that the rear side surface is arranged in circular ring manner on the external surface of the dispensing side.

For this purpose, the rear side surface includes a circular structure, designed, for example, as an elevation or a depression at least regions of which are implemented as negative form of the external surface of the dispensing side. In one embodiment, the circular structure is situated in the area of the edges of the seal and thus also in the area of the edges of the device that are stabilised by the external walls of the container such that the seal touches against the device in form-fitting manner in one embodiment in the area of the seal edges, which ensures increased mechanical strength of the device, in one embodiment upon canting and the contact angle of the device against the bone canal being non-perpendicular. Moreover, the seal touching against the external surface of the dispensing side in circular manner leads to a broad-ranging contact over a wide part of the seal and thus increases the mechanical strength of the seal and of the device as a whole. In one embodiment, the circular structure of the rear side surface does not extend into the area of the dispensing opening such that the mechanical stress during the pressing against the bone canal is exerted mainly onto the stable edges of the device and not onto the less stable dispensing opening.

A further embodiment relates to a device for the application of bone cement into the cancellous bone of a bone canal, including a container, in which a bone cement can be stored, whereby the container includes, on one end, a dispensing side with a dispensing opening protruding from the dispensing side for dispensing the bone cement from the container into the bone canal, a seal for sealing a bone canal opening in order to apply the bone cement into the bone canal at sufficient application pressure, whereby the seal is reversibly connected in form-fitting and/or force-locking manner to the dispensing opening, characterised in that the seal is arranged in form-fitting manner, at least in part, on an external surface of the dispensing side.

The device includes a container. A container [shall be understood] to be a tubular vessel that includes an internal space and a container wall surrounding the internal space. The container possesses a cross-section perpendicular to a longitudinal axis. According to one embodiment, the cross-section of the container can take any shape. For example, the cross-section can be designed to be oval, square, pentagonal, hexagonal, irregular or circular.

The container is preferred to include a cylindrical geometry with a rotationally symmetrical axis with a round cross-section. The geometry allows for good handling by the user and the absence of edges reduces the risk of wedging during the use of the device. According to one embodiment, the container can consist of a broad range of materials or combinations of materials. For example, the container can consist of plastic material. In one embodiment, the plastic material is a transparent plastic material as this allows the user to check by eye the proper function of the device during a use thereof.

The container includes, on an end, in one embodiment on an axial end, a dispensing side that rounds off the container. The dispensing side includes a dispensing opening through which the bone cement can be conveyed from the internal space of the container, whereby the dispensing opening extends axially in the direction of the side of the dispensing side facing away from the internal space of the container. The protruding dispensing opening permits the reversible attachment of aids, such as for example a snorkel or a seal, which are useful or required during the application of the bone cement.

The attachment of the seal can be implemented in a variety of ways. In one embodiment, the dispensing opening includes an internal thread that acts in concert with an external thread of the seal in order to establish a form-fitting and/or force-locking connection. In a further embodiment, the dispensing opening includes an external thread that acts in concert with an internal thread of the seal in order to establish a form-fitting and/or force-locking connection. In a further embodiment, the dispensing opening and the seal are connected to each other by using a bayonet connection. In a further embodiment, the seal includes a recess that acts in concert in form-fitting and/or force-locking manner with the dispensing opening protruding from the dispensing side in order to attach the seal. In a further, preferred, embodiment, the internal diameter of the seal canal corresponds essentially to the external diameter of the dispensing opening, such that the seal can be connected to the dispensing opening in form-fitting and/or force-locking manner by inserting the dispensing opening into the seal canal.

The seal is arranged on an external surface of a dispensing side, at least partly, in form-fitting manner. The external surface of the dispensing side is the side of the dispensing side that faces the seal and faces away from the internal space. A form-fitting arrangement shall be understood to mean that the seal, in one embodiment a rear side surface situated opposite from the sealing surface, and the external surface of the dispensing side are in direct physical contact, at least over regions thereof. In the embodiment, the seal is in direct physical contact both with the dispensing side opening, for attaching the seal to the device, as well as with the external surface of the dispensing side.

One advantage of the seal touching against the dispensing side surface in form-fitting manner is that the seal possesses a high mechanical strength when pressed against the bone canal. Touching against the dispensing side provides for a more extensive distribution of force onto the seal when the device is pressed against the bone canal than would be the case upon attachment of the seal just in spots, for example if there is any physical contact exclusively with the dispensing opening. In one embodiment, the form-fit allows the connection between seal and device to be mechanically stressed to a lesser degree. This does not only increase the mechanical strength of the seal, but also the mechanical strength of the device as a whole, in one embodiment of the attachment site of the seal, such as, for example, the dispensing opening. This is advantageous in one embodiment upon canting of the seal in the bone canal which may cause the device to fracture due to the high requisite contact pressure against the bone if the mechanical stability is low. The increased mechanical strength of the seal and of the device as a whole allows the surgeon to exert a higher contact pressure against the bone canal, which allows for complete sealing of the bone canal opening, in one embodiment if the contact angle of the device against the bone is not perpendicular.

A further embodiment of the device is characterised in that the application pressure is sufficient to allow the bone cement to penetrate into the cancellous bone by at least 2 mm, in one embodiment between 3 to 5 mm deep.

A penetration depth of less than 2 mm leads to the bone cement, and the prosthesis, in one embodiment the hip prosthesis, fixed with the bone cement coming loose due to the stress of it being in use, in one embodiment during walking motions of the patient. This leads to secondary surgeries that could be prevented or at least delayed in time at a penetration depth of more than 2 mm.

If the area of the affixed prosthesis were to become infected directly postoperatively or over the course of time, the prosthesis needs to be replaced. This is done, inter alia, by completely removing the bone cement used for fixation along with the region of the cancellous bone into which the bone cement has penetrated. In order not to have to remove excessive fractions of the cancellous bone and to be able to use part of the residual cancellous bone again for improved adhesion of bone to bone cement in the subsequent fixation of the new prosthesis, it is preferred not to allow the bone cement to penetrate into the cancellous bone by more than 5 mm.

A further embodiment of the device is characterised in that the seal includes a rear side surface opposite from the sealing surface, whereby the rear side surface is arranged in form-fitting manner on the external surface of the dispensing side.

The form-fitting contact between the rear side surface and the external surface of the dispensing side can be implemented in a variety of ways. In an embodiment, at least 10 area-% of the rear side surface are arranged in form-fitting manner on the external surface of the dispensing side. Area-% of the rear side surface shall be understood to be the fraction of the rear side surface which touches against the external surface of the dispensing side in form-fitting manner, relative to the total area of the rear side surface. The higher this fraction, the higher is the mechanical strength of the seal and thus of the device as a whole as well.

A further embodiment of the device is characterised in that at least 20 area-% of the rear side surface are arranged on the external surface of the dispensing side.

Up to 100 area-% of the rear side surface can be arranged on the external surface of the dispensing side.

The rear side surface, in one embodiment the part of the rear side surface arranged in form-fitting manner on the external surface of the dispensing side, can be implemented in a variety of ways. In an embodiment, the rear side surface is implemented to be planar. In a further embodiment, the entire rear side surface is implemented as negative form of the external surface of the dispensing side such that the entire rear side surface or at least a part of the rear side surface is arranged in form-fitting manner on the external surface of the dispensing side.

A further embodiment of the device is characterised in that the rear side surface is arranged in circular ring manner on the external surface of the dispensing side.

For this purpose, the rear side surface includes a circular structure, designed, for example, as an elevation or a depression at least regions of which are implemented as negative form of the external surface of the dispensing side. In one embodiment, the circular structure is situated in the area of the edges of the seal and thus also in the area of the edges of the device that are stabilised by the external walls of the container such that the seal touches against the device in form-fitting manner in one embodiment in the area of the seal edges, which ensures increased mechanical strength of the device, in one embodiment upon canting and the contact angle of the device against the bone canal being non-perpendicular. Moreover, the seal touching against the external surface of the dispensing side in circular manner leads to a broad-ranging contact over a wide part of the seal and thus increases the mechanical strength of the seal and of the device as a whole. In one embodiment, the circular structure of the rear side surface does not extend into the area of the dispensing opening such that the mechanical stress during the pressing against the bone canal is exerted mainly onto the stable edges of the device and not onto the less stable dispensing opening.

A further embodiment is related to a method for applying bone cement into the cancellous bone of a bone canal by using a device according to any one of the preceding embodiments, including at least the following:
a) providing the bone cement inside the device;
b) pressing the device onto a bone canal opening such that the bone canal is sealed by a seal during the application of the bone cement;
c) dispensing the bone cement from the device into the bone canal;
   whereby the seal is being pressed essentially onto a surface of the bone and/or onto a cut surface of the bone.

The provision of the bone cement inside the device, in one embodiment inside a container of the device, in process a) can take place in a variety of ways. In an embodiment of the method, the bone cement is mixed outside of the device and is moved into the container subsequently. In a further, preferred embodiment of the method, the bone cement is mixed inside the device, in one embodiment inside the container, such that the user does not need to transfer the bone cement after mixing.

Prior to pressing it onto the bone canal opening in process b), the device is fitted with a seal. In this context, the seal can be reversibly attached to the device, in one embodiment to a dispensing opening of the device, at various points in time. In an embodiment of the method, the seal is already attached to the device, before the bone cement is provided inside the container in process a). In a further embodiment, the seal is being attached to the device only after the provision in process a), but before the device is pressed against the bone canal opening in process b).

The device is being pressed against the bone canal in process c) in appropriate manner such that no bone cement can exit from the bone canal, in one embodiment between seal and bone canal opening. By this means, continued conveyance of bone cement into the bone canal builds up a sufficient application pressure for the bone cement to be introduced, at least in part, into the cancellous bone of the bone. This provides for good bonding of bone cement and bone and thus for increased mechanical strength of the affixed prosthesis, in one embodiment hip prosthesis. In order to attain a sufficiently high application pressure for at least partial penetration of the bone cement into the cancellous bone, the device needs to be pressed against the bone canal opening at a contact pressure that corresponds at least to the application pressure to be attained.

The method is characterised in that the seal is being pressed essentially onto a surface of the bone and/or onto a cut surface of the bone in process b).

By this means, the seal is essentially not in physical contact with the cancellous bone, by means of which the bone cement attains free access to the cancellous bone during the application. This enables at least partial introduction of the bone cement into the cancellous bone.

A seal with a concave sealing surface, which in one embodiment is arranged at least in part in form-fitting manner on an external surface of the dispensing side of the device, enables the device to be pressed essentially onto a surface of the bone and/or onto a cut surface of the bone.

An embodiment of the method is characterised in that the bone canal is being filled essentially completely with bone cement in process c). This is made possible, in one embodiment, through a seal having a concave sealing surface. Referring to seals that reach deeply into the bone canal since the sealing surface is non-concave, there is a need after the application at high application pressure and after removal of the device from the bone canal opening to subsequently fill the hollow space that arises due to the seal and is not filled with bone cement in a further application process, this one without the seal. Referring to essentially incompletely filled bone canals, the affixation of a prosthesis can be associated with entrapment of air between prosthesis and bone cement, which worsens the mechanical fixation of the prosthesis.

Moreover, the bone cement cannot reach all areas of the cancellous bone if the bone canal is incompletely filled, which reduces the adhesion of bone cement to bone.

According to one embodiment, a bone cement shall be understood to be a substance that is well-suited to provide a stable connection between artificial joints, such as, for example, hip and knee joints, and bone tissue in the realm of medical technology. In one embodiment, bone cements are polymethylmethacrylate bone cements (PMMA bone cements). PMMA bone cements have been used in medical applications for a long period of time and are based on the pioneering work of J. Charnley (cf. Charnley, J. Anchorage of the femoral head prosthesis of the shaft of the femur. J. Bone Joint Surg. 1960; 42, 28-30). In this context, PMMA bone cements can be produced from a bone cement powder as first starting component and a monomer liquid as second starting component. If the composition is appropriate, the two starting components separated from each other can be stable on storage. When the two starting components are contacted to each other, the polymer components of the bone cement powder are generated by swelling forming a plastically deformable bone cement, which is also referred to as bone cement dough. A polymerisation of the monomer by radicals is initiated in this context. Upon advancing polymerisation of the monomer, the viscosity of the bone cement increases until the bone cement solidifies completely.

According to one embodiment, a bone cement powder shall be understood to be a powder, which includes at least one particulate polymethylmethacrylate and/or one particulate polymethylmethacrylate copolymer. Examples of copolymers include styrene and/or methylacrylate. In an embodiment, the bone cement powder can include, in addition, a hydrophilic additive that supports the distribution of the monomer liquid within the bone cement powder. In a further embodiment, the bone cement powder can include, in addition, an initiator that initiates the polymerisation. In a further embodiment, the bone cement powder can include, in addition, a radiopaquer. In yet a further embodiment, the bone cement powder can include, in addition, pharmaceutically active substances, such as, for example, antibiotics.

In one embodiment, the bone cement powder includes at least one particulate polymethylmethacrylate and/or one particulate polymethylmethacrylate copolymer, an initiator, and a radiopaquer or it consists of the components. In one embodiment, the bone cement powder includes at least one particulate polymethylmethacrylate and/or one particulate polymethylmethacrylate copolymer, an initiator, a radiopaquer, and a hydrophilic additive or it consists of the components. In one embodiment, the bone cement powder includes at least one particulate polymethylmethacrylate and/or one particulate polymethylmethacrylate copolymer, an initiator, a radiopaquer, a hydrophilic additive, and an antibiotic or it consists of the components.

According to one embodiment, the particle size of the particulate polymethylmethacrylate and/or of the particulate polymethylmethacrylate copolymer of the bone cement powder can correspond to the sieved fraction of less than 150 μm, in one embodiment, of less than 100 μm.

According to one embodiment, the hydrophilic additive can be implemented to be particulate and/or fibrous. In a further embodiment, the hydrophilic additive can be poorly soluble, in one embodiment insoluble, in methylmethacrylate. In a further embodiment, the hydrophilic additive can possess an absorption capacity of at least 0.6 g methylmethacrylate per gram of hydrophilic additive. In a further embodiment, the hydrophilic additive can include a chemical substance with at least one OH group. In this context, one embodiment can preferably provide the hydrophilic additive to possess covalently bound OH groups on its surface. Examples of the preferred hydrophilic additives can be additives selected from the group including cellulose, oxycellulose, starch, titanium dioxide, and silicon dioxide, whereby pyrogenic silicon dioxide is preferred in one embodiment. In an embodiment, the particle size of the hydrophilic additive can correspond to the sieved fraction of less than 100 μm, in one embodiment, of less than 50 μm, and in one embodiment of less than 10 μm. The hydrophilic additive can be present in an amount of up to 0.1 to 2.5 wt. %, relative to the total weight of the bone cement powder.

According to one embodiment, the initiator can contain dibenzoyl peroxide or consist of dibenzoyl peroxide.

According to one embodiment, a radiopaquer shall be understood to be a substance that allows the bone cement to be visualised on diagnostic radiographs. Examples of radiopaquers can include barium sulfate, zirconium dioxide, and calcium carbonate.

According to one embodiment, the pharmaceutically active substance can include one or more antibiotics and, if applicable, added cofactors for the one or more antibiotics. In one embodiment, the pharmaceutically active substance consists of one or more antibiotics and, if applicable, added cofactors for the one or more antibiotics. Examples of antibiotics include, inter alia, gentamicin, clindamycin, and vancomycin.

According to one embodiment, the monomer liquid can include the methylmethacrylate monomer or consist of methylmethacrylate. In an embodiment, the monomer liquid includes, aside from the monomer, an activator that is dissolved therein, such as, for example, N,N-dimethyl-p-toluidine, or consist of methylmethacrylate and N,N-dimethyl-p-toluidine.

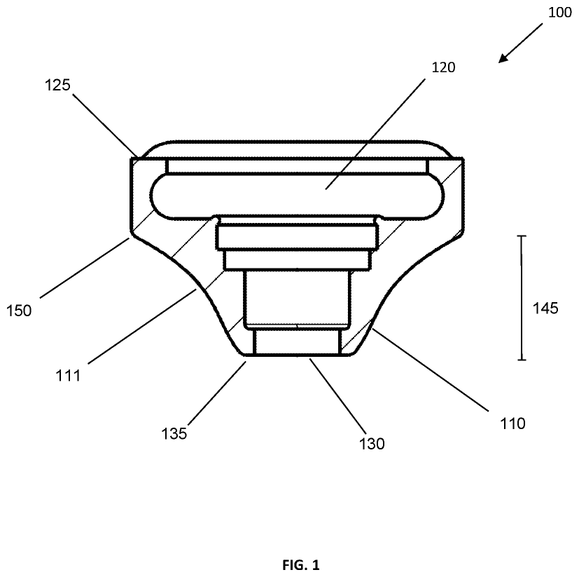

FIG. 1 illustrates a seal 100 for sealing a bone canal for use in the course of an application of a bone cement from a device. In order to apply the bone cement from the device through the seal 100 into the bone canal, the seal 100 includes a sealing canal 120. The sealing canal 120 is a fluid-conducting, tube-like connection between a sealing surface 110 facing the bone canal during the application of the bone cement and a rear side surface 125 through which the bone cement can be conveyed and which is opposite from the sealing surface 110 and faces the device.

The sealing surface 110 includes a seal opening 130 into which the seal canal 120 merges and through which the bone cement can be conveyed into the bone canal. The seal opening 130 is surrounded by a seal opening area 135 that extends radially about the seal opening 130. During the application of the bone cement into the bone canal, the seal opening 130 and the seal opening area 135 protrude into the bone canal.

The seal 100, in one embodiment the sealing surface 110, is being pressed against the bone canal before and during the application of the bone cement. This prevents the bone cement from inadvertently leaking from the bone canal, whereby a sufficiently high application pressure is reached upon the continued application of bone cement such that the bone cement is being introduced, at least in part, into a cancellous bone of the bone canal. The penetration of the bone cement into the cancellous bone increases the adhesion force between bone and bone cement and thus provides for improved mechanical fixation of a prosthesis that is being affixed in the bone canal by using the bone cement.

The sealing surface 110 surrounding the seal opening area 135 is designed to be concave. Due to the concave design, a sealing surface centre 111 recedes with respect to the seal opening area 135 and the edges 150 of the sealing surface 110. The sealing surface 110 being concave allows for good sealing of a bone canal by using the seal 100, whereby the sealing surface 110 includes only a relatively small penetration depth during the application into the bone canal due to a relatively small maximum design height 145. One advantage of the penetration depth being small is that the bone canal can be filled essentially completely with bone cement in a single filling process. Moreover, the sealing surface 110 being concave allows for at least partial penetration of the bone cement into the cancellous bone of the bone canal which is situated predominantly in the area of the bone canal opening.

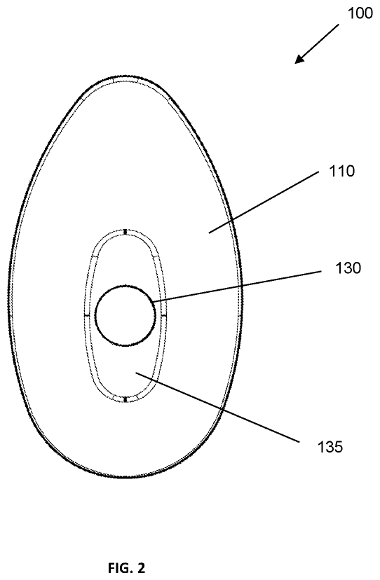

FIG. 2 illustrates the seal 100 from FIG. 1 in the form of a top view onto the sealing surface 110. The seal opening 130 is surrounded by seal opening area 135. The seal opening 130, and thus the seal opening area 135 as well, is arranged in the centre in the sealing surface 110. The seal opening area 135 and the entire seal 100 include an oval cross-sectional surface. One advantage of the oval cross-section is that this allows a multitude of different bone canal sizes and bone canal shapes to be sealed.

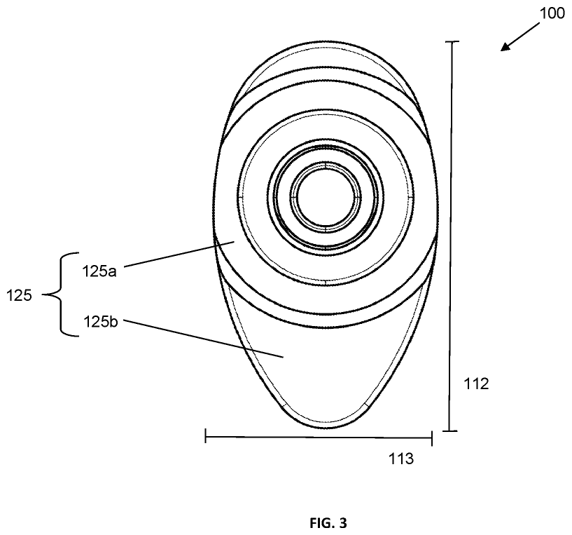

FIG. 3 illustrates the seal 100 from FIGS. 1 and 2 in the form of a top view onto the rear side surface 125. The rear side surface 125 includes a circular rear side surface region A 125a and a further rear side surface region B 125b, whereby the rear side surface region A 125a is suitable for being arranged in a form-fit on a device for application of a bone cement. The circular rear side surface region A 125a includes the entire width 113 of the oval seal 100, in one embodiment of the oval rear side surface 125. The rear side surface region B 125b is not suitable for being arranged in a form-fit on the device, but rather extends radially, in one embodiment along a length 112 of the seal 100, beyond a cross-section of the device. This simplifies the handling of the seal 100 to a user, in one embodiment an attachment and detachment of the seal 100 to and from the device.

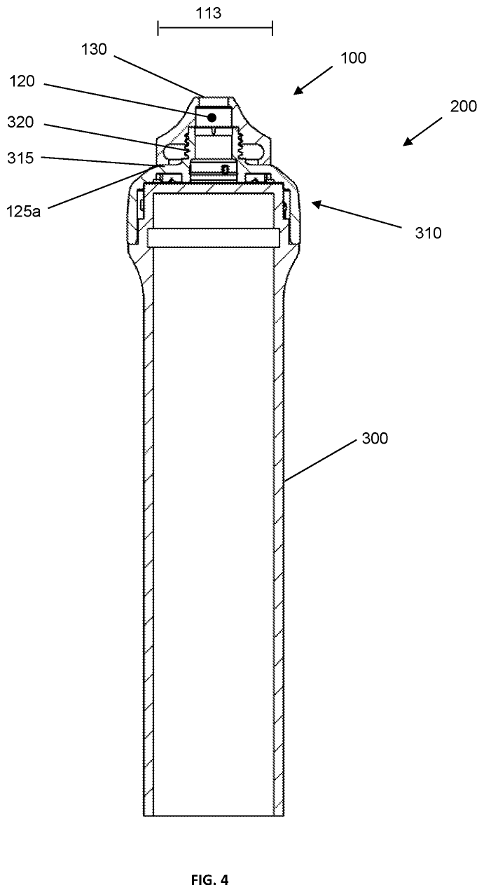

FIG. 4 illustrates a device 200 for application of a bone cement, including the seal 100 from FIGS. 1 to 3. The device 200 includes a container 300, in which a bone cement can be stored. The container 300 includes, on an axial end, a dispensing side 310 with a dispensing opening 320 that protrudes axially from the dispensing side 310. The dispensing opening 320 serves for dispensing the bone cement from the container 300 into a bone canal. The dispensing opening 320 is introduced into the seal canal 120, whereby the seal 100 is attached to the container 300 in a form-fitting and/or force-locking manner and, simultaneously, the container 300 is connected to the seal opening 130 in a fluid-conveying manner with respect to the bone cement. The connection between the dispensing opening 320 and the seal 100 is designed appropriately such that the bone cement can be applied from the container 300 via the dispensing opening 320 and through the seal canal 120 without concurrent detachment of the seal 100 from the dispensing opening 320. In further embodiments not illustrated here the seal 100 is connected to the container 300 not through the dispensing opening 320 being inserted into the seal canal 120, but rather, for example, through a threaded connection between dispensing opening 320 and seal canal 120.

The seal 100, in one embodiment the circular rear side surface region A 125a of the seal 100 in FIG. 3, is arranged in form-fitting manner on an external surface 315 of the dispensing side of the container 300. Since the circular rear side surface region A 125a includes the entire width 113 of the seal 100, in one embodiment of the oval rear side surface 125, the seal 100 being in a form-fit increases the mechanical stability of the seal 100 itself as well as of the entire device 200, in one embodiment of the connection of seal 100 and container 300. This permits the exertion of a sufficiently high contact pressure of the device 200 against a bone canal opening in order to effect partial introduction of the bone cement into the cancellous bone.

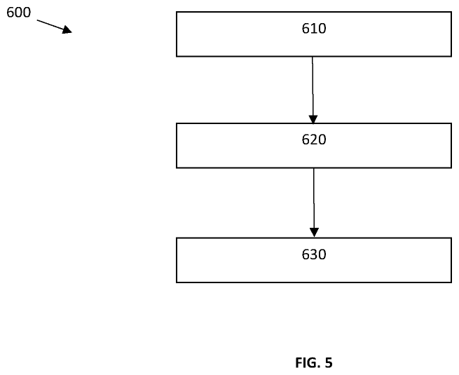

FIG. 5 illustrates a flow diagram containing the processes 610 to 630 of a method 600 for the application of a bone cement into the cancellous bone of a bone canal by using the device 200 including the seal 100. In a process 610, the bone cement is provided inside the container 300. In an embodiment of the method 600, the bone cement is provided in the container 300 by introducing into the container 300 a bone cement that can be used for surgical purposes and is mixed from the corresponding starting materials. In one embodiment, the bone cement is provided by mixing the corresponding starting materials inside the container 300. The latter is preferred, since the bone cement thus provided can be used for a short period of time only, for example in a time window of up to 5 minutes after being provided, before a curing is progressed too far and it can no longer be applied.

In a process 620, the device 200 is pressed onto a bone canal opening such that the bone canal is sealed by the seal 100. Due to the material of the seal being relatively soft, this may be associated with a deformation, in one embodiment of the sealing surface 110. Due to the sealing surface 110 being concave, the seal 100 is essentially being pressed onto a surface of the bone and/or onto a cut surface of the bone generated by the surgeon in preparation of the surgery, rather than on an internal wall of the bone canal in the vicinity of the bone canal opening, in one embodiment not the cancellous bone of the bone canal.

In a process 630, the bone cement is being dispensed from the device 200 into the bone canal, for example via a dispensing plunger that can be axially shifted in the container 300. Since the seal 100 is essentially being pressed onto the top side of the bone and/or onto the cut surface of the bone, rather than against the cancellous bone of the bone canal, the cancellous bone stays spatially accessible to the bone cement while the bone canal is being filled. Due to the bone canal being sealed by using the seal 100, continued application of the bone cement into the bone canal leads to a sufficiently high application pressure being reached such that the bone cement is introduced, at least in part, into the cancellous bone. This increases the adhesion between bone and bone cement and thus also the mechanical stability of a prosthesis that is affixed to the bone by using the bone cement. Moreover, due to the sealing surface 110 being concave, the seal 100 penetrates into the bone canal only to the extent such that the bone canal can be filled essentially completely with the bone cement in process 630, whereby no additional filling process is required before the prosthesis to be affixed is inserted into the filled bone canal. This is of advantage especially in the context of time-sensitive surgeries and also provides for a more homogeneous consistency of the bone cement, which is reflected in a higher mechanical stability of the bone cement.

The features disclosed in the claims, description and figures can be essential for the implementation of various embodiments of the claimed invention both separately and in any combination thereof. The features disclosed for the seal and the device shall be considered to be disclosed for the method as well, and vice versa.

Although specific embodiments have been illustrated and described herein, it will be appreciated by those of ordinary skill in the art that a variety of alternate and/or equivalent implementations may be substituted for the specific embodiments illustrated and described without departing from the scope of the present embodiments. This application is intended to cover any adaptations or variations of the specific embodiments discussed herein. Therefore, it is intended that these embodiments be limited only by the claims and the equivalents thereof.

1. A seal for sealing a bone canal opening during the application of bone cement into a cancellous bone of a bone canal, wherein the seal comprises a concave sealing surface for sealing the bone canal.

2. The seal according to claim 1, wherein the seal comprises a seal canal, whereby the seal canal can be reversibly connected in form-fitting and/or force-locking manner to a dispensing opening of a device providing the bone cement in order to apply the bone cement from the device out of a seal opening at an end of the seal canal that faces the sealing surface into the bone canal.

3. The seal according to claim 1, wherein the seal opening is arranged in the center of the sealing surface.

4. The seal according to claim 2, wherein the sealing surface comprises a design height, whereby a maximum design height of the seal is evident adjacent to a seal opening area and the design height decreases from the seal opening area in the direction of the edges of the sealing surface according to a concave function.

5. The seal according to claim 4, wherein the concave function comprises a largest slope adjacent to the seal opening area, and in that the slope decreases steadily in the direction of the edges of the sealing surface.

6. The seal according to claim 4, wherein the maximum design height of the sealing surface is in the range of 5 to 20 mm.

7. The seal according to claim 1, wherein the sealing surface comprises an oval cross-sectional surface having a length in the range of 60 to 80 mm and a width in the range of 25 to 45 mm.

8. The seal according to claim 1, wherein the seal comprises a plastic material, in particular a thermoplastic resin, having a Shore hardness of 25 to 50 ShoreA, preferably consists of a plastic material, in particular a thermoplastic resin, having a Shore hardness of 25 to 50 ShoreA.

9. A device for the application of bone cement into the cancellous bone of a bone canal, comprising:
a container, in which a bone cement can be stored;
whereby the container comprises, on one end, a dispensing side with a dispensing opening protruding from the dispensing side for disposing the bone cement from the container into the bone canal;
wherein the dispensing opening is reversibly connected in form-fitting and/or force-locking manner to a seal for sealing a bone canal opening during the application of bone cement into a cancellous bone of a bone canal in order to apply the bone cement into the bone canal at sufficient application pressure, wherein the seal comprises a concave sealing surface for sealing the bone canal for sealing a bone canal opening.

10. The device according to claim 9, wherein the application pressure is sufficient to allow the bone cement to penetrate into the cancellous bone by at least 2 mm.

11. The device according to claim 9, wherein the application pressure is sufficient to allow the bone cement to penetrate into the cancellous bone between 3 to 5 mm deep.

12. The device according to claim 9, wherein the seal is arranged on an external surface of a dispensing side, at least partly, in form-fitting manner.

13. The device according to claim 9, wherein the seal comprises a rear side surface opposite from the sealing surface, whereby the rear side surface is arranged on the external surface of the dispensing side in form-fitting manner.

14. The device according to claim 13, wherein at least 20 area-% of the rear side surface are arranged on the external surface of the dispensing side.

15. The device according to claim 13, wherein the rear side surface is arranged in circular ring manner on the external surface of the dispensing side.

16. A method for applying bone cement into the cancellous bone of a bone canal by means of a device according to claim 9, comprising:
a) providing the bone cement inside the device;
b) pressing the device onto a bone canal opening such that the bone canal is sealed by a seal during the application of the bone cement;
c) dispensing the bone cement from the device into the bone canal;
whereby the seal is being pressed essentially onto a surface of the bone and/or onto a cut surface of the bone.
